(19) United States
(12) Patent Application Publication
(10) Pub. No.: US 2006/0147423 A1
(43) Pub. Date: Jul. 6, 2006

(54) Transungual device

(76) Inventors: Jean-Yves Legendre, Paris (FR); Roberto Cavazzuti, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/295,570

(22) Filed: Dec. 7, 2005

Related U.S. Application Data
(60) Provisional application No. 60/634,549, filed on Dec. 10, 2004.

(30) Foreign Application Priority Data
Dec. 7, 2004 (FR) 04 52892

(51) Int. Cl. A61K 35/74

(52) U.S. Cl. 424/93.4; 424/780

(57) ABSTRACT

Disclosed herein is a transungual device comprising at least one active agent chosen from extracts of a non-fruiting, non-photosynthetic filamentous bacterium, dialkyl sulphones, and mixtures thereof. Also disclosed herein is a method for the cosmetic treatment of nails comprising applying the transungual device to the nails.

This application claims benefit of U.S. Provisional Application No. 60/634,549, filed Dec. 10, 2004, the contents of which are incorporated herein by reference. This application also claims benefit of priority under 35 U.S.C. § 119 to French Patent Application No. 04 52892, filed Dec. 7, 2004, the contents of which are also incorporated herein by reference.

Disclosed herein is a transungual device comprising at least one active agent chosen from extracts of a non-fruiting, non-photosynthetic filamentous bacterium, dialkyl sulphones, and mixtures thereof.

It is well known that finger and toe nails frequently exhibit structural defects and defects of consistency, it being possible for said defects to be of various origins, and in particular related to the individual's own metabolism and to the individual's lifestyle, eating habits, age, state of fatigue, and/or state of overwork.

These defects may also appear due to the effect of actions that erode, subsequent, for example, to prolonged or repeated exposure to detergents, to solvents, to chemical products, for example, household products, to hot or cold and wet or dry atmospheres, and/or to UV radiation.

These structural defects or defects of consistency have the effect of making the surface of the nails unattractive, which may be a source of considerable inconvenience and embarrassment.

Products comprising active agents capable of reinforcing the nails, which are generally applied topically in the form of gels or lotions, are known; however, the diffusion of the active principle through the surface of the nail is weak and the treatment time may be long. In addition, these forms of topical dosage do not always allow the active principle to be kept in contact with the nail for a prolonged period of time.

Another known topical form is a nail varnish, but treatment of the nails with a varnish is quite restrictive since it is repetitive and requires maintenance of the nail before each application.

The present inventors have discovered that a specific active agent chosen from extracts of a non-fruiting, non-photosynthetic filamentous bacterium and/or dialkyl sulphones is capable of diffusing efficiently through the nail by means of a transungual device.

Thus, disclosed herein is a transungual device comprising at least one active agent chosen from extracts of a non-fruiting, non-photosynthetic filamentous bacterium, dialkyl sulphones, and mixtures thereof.

Such a device makes it possible, after application to the nails, to strengthen the nails (such as soft nails), for example, to harden the nails, to increase their resistance to breaking and/or to splitting, to increase the thickness of the nails, and/or to stimulate the growth and/or regrowth of the nails, and thus, to improve their overall appearance.

The transungual device also makes it possible to limit the trans epidermal water loss of the nails and/or to improve the moisturizing of the nails and the penetration of the active agent in the nails. As used herein, the term “trans epidermal water loss” means the water percentage that crosses the keratinic material of the nail and evaporates at the surface.

In addition to improving the bioavailability and/or the contact of the active agent with the nail, the device disclosed herein makes it possible to incorporate a large amount of active agent, greater than that of a nail varnish, in which the content of active agent can be limited by the problems of solubility of the active agent in the solvent medium.

Such active agents are described, for example, in International Application Publication No. WO 02/056858 and European Patent No. 0 761 204 for the bacterial extract and French Patent No. 0 216 651 for the dialkyl sulphone. However, none of these documents discloses the use of such active agents in the form of a transungual device for strengthening the nails and improving the general condition of the nail.

Also disclosed herein is a method for strengthening the nails, for example, hardening the nails, increasing their resistance to breaking and/or to splitting, increasing the thickness of the nails, and/or stimulating the growth and/or regrowth of the nails, comprising applying to the nails a transungual device comprising at least one active agent chosen from extracts of a non-fruiting, non-photosynthetic filamentous bacterium, dialkyl sulphones, and mixtures thereof.

Further disclosed herein is a method for limiting the trans epidermal water loss of nails, to improve the moisturizing of the nails, and/or to improve the penetration of the active agent in the nails, comprising applying to the nails a transungual device comprising at least one active agent chosen from extracts of a non-fruiting, non-photosynthetic filamentous bacterium, dialkyl sulphones, and mixtures thereof.

Still further disclosed herein is a method for cosmetically treating nails comprising applying to the nails a transungual device comprising at least one active agent chosen from extracts of a non-fruiting, non-photosynthetic filamentous bacterium, dialkyl sulphones, and mixtures thereof.

Active Agent

The active agent may be chosen from extracts of a non-fruiting, non-photosynthetic filamentous bacterium, dialkyl sulphones, and mixtures thereof.

Bacterial Extract

Suitable bacterial extracts for use in the context of the present disclosure may be prepared from non-photosynthetic filamentous bacteria as defined according to the classification of Bergey's Manual of Systematic Bacteriology (vol. 3, sections 22 and 23, 9^{th} edition, 1989), for example, bacteria belonging to the order Beggiatoales, such as bacteria belonging to the Beggiatoa, Vitreoscilla, Flexithrix, and Leucothrix genera.

Non-limiting examples of suitable bacteria include:
Vitreoscilla filiformis (ATCC 15551),
Vitreoscilla beggiatoides (ATCC 43181),
Beggiatoa alba (ATCC 33555),
Flexithrix dorotheae (ATCC 23163),
Leucothrix mucor (ATCC 25107), and
Sphaerotilus natans (ATCC 13338).

In at least one embodiment, an extract of Vitreoscilla filiformis (ATCC 15551) may be used.

As used herein, the term “bacterial extract” is intended to mean an extract of the bacterial biomass or any active fraction of said extract, for example:

(i) bacterial cells isolated from the culture medium, which have been concentrated, for example by centrifugation (non-stabilized cell extract);

(ii) bacterial cells that have been concentrated (i), and then subjected to an operation comprising rupturing the bacterial cell envelopes by any means known to those skilled in the art, such as the action of ultrasound or autoclaving (stabilized cell extract). As used herein, the term “envelopes” is intended to mean bacterial walls and, optionally, the underlying membranes; or

(iii) the supernatant obtained by filtration of the stabilized cell extract (ii), or any active fraction of said extract. As used herein, the term “active” fraction of said extract is intended to mean any fraction capable of strengthening and/or improving the keratin materials according to the invention.

This active fraction can be obtained by conventional fractionation methods, such as extraction in the presence of a solvent, selective precipitation, and tangential-flow ultrafiltration (TFU), for example.

These extracts or fractions may be conserved, for example, by freezing said extracts or said fractions, and may be used after thawing.

In the remainder of the description, reference will more simply be made to bacterial “cell extract” ((i) and (ii)) or to “supernatant” of said extract (iii).

In addition, in at least one embodiment of the present disclosure, a bacterial “cell extract” (in the form (i) or (ii)) or an active fraction of said extract may be used.

To prepare the bacterial extract according to the present disclosure, said bacteria can be cultured according to the methods known to those skilled in the art. Reference may be made, for example, to the description in International Application Publication No. WO-A-93/00741. A cell extract from which the supernatant can be separated, for example, by filtration and centrifugation, is obtained. The extract may be used in aqueous form or in lyophilized form. This protocol is described in greater detail in Example 1 of the present disclosure

This bacterial extract may be refractionated and used pure or diluted to various concentrations.

Dialkyl sulphone

Suitable dialkyl sulphones may include those of formula (I):
R₁—SO₂—R₂ (I)

in which R₁ and R₂, which may be identical or different, are chosen from linear or branched alkyl groups comprising from 1 to 4 carbon atoms. In one embodiment, these dialkyl sulphones may be used in a cosmetic nail care composition applied to the nails so as to promote nail growth.

The alkyl group of R₁ and R₂ may be independently chosen from linear or branched alkyl groups comprising from 1 to 4 carbon atoms, such as methyl, ethyl, isopropyl, n-propyl, n-butyl, isobutyl, and tert-butyl groups.

In at least one embodiment of the present disclosure, R₁ and R₂ may be identical. For example, R₁ and R₂ may both be methyl groups, such that the compound of formula (I) is dimethyl sulphone or methylsulphonylmethane (MSM).

The compounds of formula (I), and in particular MSM, are known compounds. MSM [67-71-0] has the advantage of being an odorless white crystalline product having a melting point of 109° C. and a boiling point of 238° C.

MSM is present in most green plants, microplankton, and algae. It is a natural compound that is found in nerve tissues, the skin, the hair, and the joints.

The active agent may be present in the device in an amount ranging from 0.1% to 90% by weight relative to the total weight of the matrix of the device, as defined below, for example, from 0.5 to 50% by weight, or from 1 to 30% by weight.

Device

As used herein, the term “transungual device” or “transungual delivery system” is intended to mean any system that allows active or passive release of the active substance, allowing it to be transferred into the matrix of the nail.

Non-limiting examples of suitable transungual devices include:

patches, i.e., any system for delivering an active agent that has a composite structure in the form of layers which, by application to the nail, ensures the release of the active product through the nail. Examples of patches include, but are not limited to:
controlled-release patches with a hydrophobic polymeric matrix, as described, for example, in French Patent No. 2 738 744;
reservoir-type patches comprising a reservoir of active substance, a diffusion membrane, and an adhesive layer;
magnetic-field-effect patches that promote penetration of the active agents into the skin, as described, for example, in French Patent No. 2 791 750;
optimized-adhesion patches comprising a layer of hydrophobic polymer attached to a support layer and containing particles of active compound, oil particles, and particles of a water-absorbing agent, as described, for example, in French Patent No. 2 761 889;
gel patches with a high water content, of hydrogel type, for example, based on at least one entity chosen from hydrocolloids, polysaccharides, proteins, polyacrylamide, polyacrylic acid, acrylic acid, starch copolymers, polyvinylpyrrolidone, and/or polyvinyl acid derivatives, and exhibiting a contact adhesion similar to that resulting from a sucker effect;
hydrophobic gel patches, also called organogels or oleogels, based on an entity chosen from polyethylene, silicones, copolymers of styrene and isoprene, and copolymers of styrene and butadiene. These systems have the advantage of being able to contain high proportions of hydrophobic compounds; and
segmented polyurethane-based gel patches (SPUG), the lipophilic or hydrophilic nature of which can be modified by modifying the polyoxyethylenated chains (see, for example, Y. Shikinami, “Adhesive polymer gels for medical uses,” Kagaku To Tokyo, 67(4), 141-150, 1993);
sheets or capsules, such as those described in French Patent Nos. 2 512 651 and 2 538 247;
composite films, such as those described in European Patent No.0 285 563 A and U.S. Patent Application Publication No. 2002/0187181. In one embodiment, use may be made of a composite film comprising a reservoir layer, in which the reservoir layer comprises a matrix formed from a silicone polymer, inside which are inclusions comprising the active aqueous phase gelled by means of at least one gelling agent (see, for example, French Patent No. 2 650 747 to L'Oréal);
solid thin films based on natural or synthetic polymers, which, when applied to the prewetted nail, dissolve in situ. Such films are described, for example, in International Application Publication Nos. WO 02/053197, WO 02/05789, WO 2004/032859, and WO 02/054997;
films comprising a film-forming agent and a hydrophilic adhesive polymer, also called patch-non-patch, as described, for example, in International Application Publication No. WO 02/30402; and
compositions based on polymers having adhesive properties capable of forming a film that is removed after drying (see, for example, European Patent Nos. 0 514 760 and 0 826 364) or an elastic and pliable solid layer that is removed by applying an adhesive fabric or plastic (see, for example, International Application Publication No. WO 93/05893); or gels or pastes which, after application to the nail, dry so as to give a film that is removed by washing, cleansing, or pulling away. Such a composition will generally be referred to as a “varnish-patch” or “peel-patch”.

However, as used herein, “transungual device” is also intended to mean all the systems or devices that increase absorption by an active mechanism (see, for example, M. R. Prausnitz et al., “Current status and future potential of transdermal drug delivery,” Nature Rev., vol. 3, 115-124, 2004), such as, for example:
pressurized or needle-free injection systems (see, for example, Burkoth T. L. et al., “Transdermal and transmucosal powdered drug delivery,” Crit. Rev. Ther. Drug Carrier Syst., 1999, 16(4), 331-84);
the use of ultrasound: sonophoresis and phonophoresis (see, for example, Joshi A. and Raje J., “Sonicated transdermal drug transport,” J. Control. Release, 83 (1), 13-22, 2002);
the use of electric currents: iontophoresis, electroporation, and microfrequency current (see, for example, Riviere J. E. and Heit M. C., “Electrically-assisted transdermal drug delivery,” Pharm. Res., 14 (6), 687-697, 1997; U.S. Pat. No. 6,148,232; and International Application Publication No. WO 03/035167);
the use of magnetic waves: magnetophoresis (see, for example, Murthy S. N., “Magnetophoresis: an approach to enhance transdermal drug diffusion,” Pharmazie, 1999 May, 54(5), 377-9); and
the use of microneedles (see, for example, McAllister D. V. et al., “Microfabricated microneedles for gene and drug delivery,” Ann. Rev. Biomed. Eng., 2, 289-313, 2000 and U.S. Pat. No. 6,451,240).

These transungual delivery systems may be formulated, according to techniques known to those skilled in the art, so as to provide the amount and the rate of diffusion suitable for the active agents present in the composition.

The transungual delivery systems may also contain the excipients that are suitable for their formulation and that are known to those skilled in the art, for example, at least one compound chosen from gelling agents, adhesive polymers, stabilizers, dyes, pigments, fillers, solvents, ions, and mixtures thereof.

In at least one embodiment, the device according to the present disclosure may be in the form of a patch comprising a flexible support and a matrix, for example, an adhesive matrix, comprising the active agent.

As used herein, the term “matrix” or “matrix adhesive layer” is intended to mean a mixture of at least one active agent as defined above, in a homogeneously dispersed or solubilized form, in a biocompatible and pressure-sensitive adhesive, which mixture may also optionally contain other additives.

In the case of a monolayer device, this matrix layer has a surface that is directly in contact with the support and a second surface that is directly in contact with the detachable outer coating to be removed when the patch is applied to the area to be treated. During use, this detachable outer coating is removed from said surface of the matrix layer, which is then applied directly to the nail.

The active principle diffuses through the adhesive and its mixtures, where appropriate, as far as the surface of the nail.

As used herein, the term “adhesive” denotes a polymer or a mixture of polymers that are chemically inert with respect to the various components of the matrix, including, for example, the at least one active agent.

The matrix may comprise at least one polymer. The polymer may be chosen from natural and synthetic polymers capable of gelling in the presence of water and of exhibiting an adhesive capacity, for example, pressure-sensitive adhesives, i.e., materials which adhere instantaneously to the skin under the application of a weak pressure. In at least one embodiment, the matrix polymer may be chosen from hydrophilic polymers.

According to one embodiment of the present disclosure, the transungual device may be a hydrophilic gel patch or hydrogel, the matrix of which comprises at least one hydrophilic polymer. Such a device makes it possible to improve the moisturizing of the nails, and thus, to increase the penetration of the active agent in the nail.

These hydrophilic polymers may be chosen from polymers and copolymers of vinylpyrrolidone, acrylic acid, the salts, derivatives and copolymers of acrylic acid, and mixtures thereof. Non-limiting examples of acrylic polymers include those sold under the trade name “Gelva” by the company Monsanto, those sold under the trade name “Duro-Tak” by the company National Starch, those sold under the name “Acronal” by the company BASF, and sodium polyacrylate, for example, the product sold under the trade name Aronvis S by the company Nihon Junyaku.

The at least one polymer may be present in the matrix in an amount ranging from 5 to 60% by weight relative to the total weight of the matrix, for example, from 10 to 40% by weight.

The matrix may, in at least one embodiment, comprise a high water content, which may, for example, range from 30 to 90% by weight relative to the total weight of the matrix.

In addition to the at least one polymer and the water, the hydrogel matrix may also comprise at least one solvent chosen from primary alcohols, such as ethanol and isopropanol, and polyols, which are compounds containing at least two hydroxyl functions. Examples of suitable polyols include, but are not limited to, glycerol, propylene glycol, butylene glycol, hexylene glycol, polyethylene glycols, dipropylene glycol, polyvinyl alcohols, and mixtures thereof. The at least one solvent may be present in the matrix in an amount ranging, for example, from 1 to 40% by weight, such as from 5 to 30% by weight, relative to the total weight of the hydrogel matrix.

The matrix may also comprise at least one hydrophilic gelling agent or hydrocolloid. These compounds may, for example, be chosen from:
gellan gum;
cellulose and its derivatives, such as carboxymethylcellulose, hydroxypropylcellulose, methylcellulose, hydroxypropylmethylcellulose, and hydroxyethylcellulose, and modified celluloses, for example, those modified by grafting of an alkyl group;
algal extracts, such as agar-agar, carrageenans, and alginates;
seed extracts, such as carob gum, guar gum, and modified guar gums, for example, those modified by grafting of an alkyl group;
plant exudates, such as gum arabic, karaya gum, gum tragacanth, and ghatti gum;
microorganism exudates, such as xanthan gum;
fruit extracts, such as pectins;
silicon derivatives, such as synthetic hectorites, for instance the products “Laponite RD” and “RDS” sold by the company Waverly, and magnesium aluminium silicates, for instance the product “Veegum” sold by the company Vanderbilt;
Polycare® products, sold by the company Rhône-Poulenc; and
mixtures thereof.

The hydrocolloid may be present in the matrix in an amount ranging from 5 to 40% by weight, for example, from 10 to 30% by weight, relative to the total weight of the matrix.

The matrix may further comprise at least one penetration accelerator, chosen, for example, from noncyclic mono- and dialcohols, ethyl acetate, butyl acetate, isopropyl myristate, fatty acids, phospholipids, terpenes, azone and derivatives, propylene glycol and glycolic derivatives, cyclodextrins, octylsalicylate, cyclopentadecanolide, polysorbates, and polyvinylpyrrolidone and derivatives. The penetration accelerator may be present in the matrix in an amount ranging from 0.01% to 10% by weight relative to the total weight of the matrix.

These agents may be chosen according to their own properties in order to modulate the adhesion and the viscosity of the matrix. The matrix may also contain moisturizers, such as glycerol, in an amount ranging from 0.1 to 30%, for example, from 1 to 20%, by weight relative to the weight of the matrix. In addition, the adhesive matrix may comprise at least one additive chosen from preserving agents, dyes, pigments, fillers, fragrances, stabilizers, polymerization initiators, aqueous solvent media, organic solvent media, gelling agents, ions, and mixtures thereof.

The support of the transungual device is the part of the patch that is visible when it is applied to the nail. The support makes it possible to maintain the active principle in contact with the nail so as to increase the diffusion of the active principle within the nail.

The support may be more or less flexible (for example, it may exhibit sufficient flexibility to adjust as well as possible to the surface of the nail, even in the case of an uneven surface) and insoluble, and may be woven or nonwoven or else may be made of a plastic chosen, for example, from polyurethane (sold, for example, under the trademarks Baydur®, Daltoflex®, Uroflex®, and Hyperlast®), polyurethane thermoplastic elastomer (sold, for example, under the trademarks Desmopan®, Estane®, Lastane®, and Texin®), SBS (styrene-butadiene-styrene) thermoplastic elastomer (sold, for example, under the trademarks Cariflex®, Kraton®, and Solprene®), SEBS (styrene-ethylene-butylene-styrene) thermoplastic elastomer (sold, for example, under the trade mark Kraton®), EVA (ethylene-vinyl acetate, sold, for example, under the trademarks Elvax®, Escorene®, and Optene®), coether ester thermoplastic elastomer (CEE TPE, sold, for example, under the trademarks Arnitel®, Hytrel®, and Riteflex®), polyethylenes, propylenes, and silicones.

The structure of the insoluble support may either be breathable or may be occlusive, i.e., comprise a substance impermeable to air and/or water vapor, so as to facilitate the penetration of the active agent into the nail.

The transungual device generally comprises a detachable outer coating. As used herein, the term “detachable outer coating” is intended to mean a protective support in direct contact with the adhesive matrix layer, which is removed just before application to the skin. It must therefore be chemically inert and impermeable with respect to the formula of the device.

Non-limiting examples of substances that may be suitable as a detachable outer coating include sheets of aluminium, papers containing a sheet of polyethylene, and siliconized polyesters.

In at least one embodiment, the transungual device may be prepared according to conventional techniques, such as coating.

According to one embodiment, a hydrogel patch may be prepared as follows. The active ingredients and the adjuvants are dissolved in water. The at least one adhesive polymer and the hydrocolloids are dissolved separately in water. The two aqueous solutions are then mixed, optionally with heating of the mixture. The latter is then coated onto its support at a thickness controlled using a coating machine. The patches are then cut out and, optionally, stored in the open air so as to allow good crosslinking of the gel.

The device according to the present disclosure may be in a form precut to the size of the nail, or in a form to be cut out before or after application thereof, according to the size and shape desired.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLES

### Example 1

### Preparation of Vitreoscilla filiformis extracts

The Vitreoscilla filiformis strain (ATCC 15551) was cultured according to the method described in International Publication No. WO-A-93/00741, which is a continuous culture method. The culturing was carried out at 26° C. for at least 48 hours until a suitable cell concentration corresponding to an optical density at 600 nm of greater than or equal to 1.5 was obtained. The strain was subcultured at 2% V/V in fresh medium for approximately 48 hours until a stable culture was obtained. A 1 liter Erlenmeyer flask containing 200 ml of fresh medium was then inoculated with 4 ml of the above culture. The culturing in the Erlenmeyer flask was carried out at 26° C. on a culture platform shaken at 100 rpm. The tank starter thus obtained was used as an inoculum for a 50 litre fermenter. Growth was carried out at 26° C., pH 7,100 rpm and pO₂≧15%.

After 30 hours of growth, the biomass was transferred into a fermenter with a usable capacity of 3000 liters, so as to be cultured under the same conditions. After 48 hours of growth, the cells were harvested continuously. The biomass was then concentrated approximately 50-fold by centrifugation. The cells obtained were then frozen as the continuous culture proceeded. These cells may be used without modification, after thawing (nonstabilized cell extract), or else may be stabilized by autoclaving at 121° C. for 20 to 40 minutes (stabilized cell extract). The cells rupture during sterilization, releasing the cytosol and agglomerating the proteins and also the walls. The product obtained was a two-phase product.

The supernatant liquid phase may be filtered at 0.22 μm so as to remove the particles (“supernatant”).

The bacterial extract, in the form of a cell extract (stabilized or nonstabilized) or of supernatant, may be used without modification (aqueous form) or can be lyophilized according to conventional techniques (lyophilized form).

### Example 2

### Patch

A gel was prepared having the following composition:

| | |
|---|---|
| Methylsulphonylmethane | 5% |
| Glycerol | 5% |
| Sodium alginate | 10% |
| Polyvinyl alcohol | 10% |
| Sodium polyacrylate (Aronvis S from Nihon Junyaku) | 5% |
| Preserving agents | 1% |
| Water q.s. for | 100% |

After mixing of the components, the gel thus obtained was coated onto a nonwoven support and then cut out so as to form a patch.

### Example 3

### Patch

A gel was prepared having the following composition:

| | |
|---|---|
| Methylsulphonylmethane | 5% |
| Glycerol | 5% |
| Sodium alginate | 8.8% |
| Polyvinyl alcohol | 11.3% |
| Acrylate copolymer | 7% |
| Preserving agents | 0.45% |
| Sodium Carboxymethylcellulose | 0.4 |
| Propylene glycol | 0.15 |
| Water q.s. for | 100% |

After mixing of the components, the gel thus obtained was coated onto a nonwoven support and then cut out so as to form a patch.

The patch of Example 3 was evaluated on 32 female subjects having fragile, flabby, breakable, and/or damaged nails.

These subjects applied the patch once a day, for at least half an hour, during 42 days on all the nails of one hand. The other hand was not treated (control). At the end of the treatment, the results were evaluated by measuring the trans epidermal water loss (TEWL), by high resolution ultrasound imaging, and by self evaluation.

1/Trans Epidermal Water Loss (TEWL)

The measurement was made with an evaporation meter Tewametre® TM210 (Courage and Khazaka) and was based on the evaluation of a vapor pressure gradient on the surface of the nail. Dependent on the relative humidity and the temperature, the TEWL value is expressed in water mass evaporated in time and using the unit of area (g/m²h).

The measurements were taken after a rest period of 20 to 30 minutes at room temperature maintained between 20 and 22° C. and a relative humidity of 40-50%.

The probe of the Tewametre was applied perpendicularly to the skin. Mean readings were taken when the values had stabilized (after 30-45 seconds).

Measurements were taken on the thumb nail of each hand before treatment, after 21 days of treatment and after 42 days of treatment.

Results:

| Control | | Treated nails | |
|---|---|---|---|
| After 21 days | After 42 days | After 21 days | After 42 days |
| of treatment | of treatment | of treatment | of treatment |
| No significant | Significant | Significant | Significant |
| variation | decrease of | decrease of | decrease of |
| of TEWL | TEWL: −11.3 | TEWL: −15% | TEWL: −28.3% |

A significant decrease of the TEWL was observed from 21 days until 42 days on the treated nails. 2/Measurement of the Nail Thickness by High Resolution Ultrasound Imaging:

The measurement was made using a Dermcup 2020 (2MT Medical-France) provided with a spindle operating at a frequency of 20MHz.

Measurements were taken on the thumb nail of each hand before the treatment (t0) and after 42 days of treatment (t42).

The visual field and the gain were maintained constant for each subject at every time of measurement. Three images were taken successively on each place of measurement and for each time of the study. The best image was recorded and analyzed. A series of 150 to 250 measurements were made to define an average thickness of the nail for the times t0 and t42.

| | Treated nails |
|---|---|
| Control | (after 42 days) |
| No significant variation | +3% increase of |
| of the nail thickness | the nail thickness |

A significant increase of the thickness of the treated nails was observed after 42 days of treatment.

3/Self Evaluation:

The subjects gave the following responses:

59% reported that the patch increased the thickness of the nail,

50% reported that the patch assisted the growth of the nails,

69% reported that the product hardened the nails, and

69% reported that the product improved the general condition of the nails.

### Example 4

### Patch

A patch was prepared having the following composition:

| | |
|---|---|
| Vitreoscilla filiformis extract of Example 1 | 5% |
| Methylsulphonylmethane | 5% |
| Glycerol | 20% |
| Polyacrylic acid (Junlon PW 150 from Nihon Junyaku) | 4% |
| Tween 80 | 1% |
| Sodium polyacrylate (Aronvis from Nihon Junyaku) | 5% |
| Tartaric acid | 1% |
| Preserving agents | 2% |
| Water q.s. for | 100% |

After mixing of the components, the gel thus obtained was coated onto a nonwoven support and then cut out so as to form a patch.

What is claimed is:

1. A transungual device comprising at least one active agent chosen from extracts of a non-fruiting, non-photosynthetic filamentous bacterium, dialkyl sulphones, and mixtures thereof.

2. The device of claim 1, wherein said bacterium belongs to a genus chosen from the Beggiatoa, Vitreoscilla, Flexithrix, and Leucothrix genera.

3. The device of claim 1, wherein said bacterium is chosen from Vitreoscilla filiformis strains.

4. The device of claim 3, wherein said bacterium is the ATCC 15551 strain.

5. The device of claim 1, wherein the bacterial extract is a cell extract or the supernatant of said cell extract.

6. The device of claim 1, wherein the bacterial extract is a cell extract or an active fraction of said cell extract.

7. The device of claim 1, wherein the dialkyl sulphones are chosen from compounds of formula (I):
R₁—SO₂—R₂ (I)
in which R₁ and R₂, which may be identical or different, are chosen from linear or branched alkyl groups comprising from 1 to 4 carbon atoms.

8. The device of claim 7, wherein the alkyl groups of R₁ and R₂ are independently chosen from methyl, ethyl, isopropyl, n-propyl, n-butyl, isobutyl, and tert-butyl groups.

9. The device of claim 7, wherein R₁ and R₂ are identical.

10. The device of claim 7, wherein R₁ and R₂ are both methyl groups and the compound of formula (I) is dimethyl sulphone or methylsulphonylmethane (MSM).

11. The device of claim 1, wherein the transungual device is in a form chosen from patches, sheets, composite films, solid films, and devices that increase the absorption by an active mechanism.

12. The device of claim 1, wherein the device is a patch chosen from controlled-release patches, reservoir patches, magnetic-field-effect patches, gel patches with a high water content (or hydrogels), and hydrophobic gel patches.

13. The device of claim 1, wherein the device is a patch comprising a flexible support and a matrix comprising the at least one active agent.

14. The device of claim 13, wherein the matrix is adhesive.

15. The device of claim 1, wherein the matrix comprises at least one polymer.

16. The device of claim 15, wherein the at least one polymer is chosen from natural and synthetic polymers exhibiting an adhesive capacity.

17. The device of claim 1, wherein the device is a hydrophilic gel patch or hydrogel, and wherein the device comprises a matrix comprising at least one hydrophilic polymer.

18. The device of claim 17, wherein the hydrophilic polymer is chosen from polymers and copolymers of vinylpyrrolidone, acrylic acid, acrylic acid derivatives, acrylic acid salts, copolymers of acrylic acid, and mixtures thereof.

19. The device of claim 15, wherein the at least one polymer is present in the matrix in an amount ranging from 5 to 60% by weight relative to the total weight of the matrix.

20. The device of claim 19, wherein the at least one polymer is present in the matrix in an amount ranging from 10 to 40% by weight relative to the total weight of the matrix.

21. The device of claim 13, wherein the matrix has a water content ranging from 30 to 90% by weight relative to the total weight of the matrix.

22. The device of claim 1, wherein the at least one active agent is present in an amount ranging from 0.1% to 90% by weight relative to the total weight of the matrix.

23. The device of claim 22, wherein the at least one active agent is present in an amount ranging from 0.5 to 50% by weight relative to the total weight of the matrix.

24. The device of claim 23, wherein the at least one active agent is present in an amount ranging from 1 to 30% by weight relative to the total weight of the matrix.

25. The device of claim 13, wherein the matrix comprises a solvent chosen from primary alcohols, polyols, polyvinyl alcohols, and mixtures thereof.

26. The device of claim 25, wherein the solvent is present in the matrix in amount ranging from 1 to 40% by weight relative to the total weight of the matrix.

27. The device of claim 26, wherein the solvent is present in the matrix in amount ranging from 5 to 30% by weight relative to the total weight of the matrix.

28. The device of claim 17, wherein the matrix comprises at least one hydrophilic gelling agent or hydrocolloid.

29. The device of claim 28, wherein the at least one hydrocolloid is present in the matrix in an amount ranging from 5 to 40% by weight relative to the total weight of the matrix.

30. The device of claim 29, wherein the at least one hydrocolloid is present in the matrix in an amount ranging from 10 to 30% by weight relative to the total weight of the matrix.

31. The device of claim 1, wherein the transungual device further comprises at least one penetration accelerator compound.

32. The device of claim 31, wherein the at least one penetration accelerator compound is chosen from noncyclic mono- and dialcohols, ethyl acetate, butyl acetate, isopropyl myristate, fatty acids, phospholipids, terpenes, azone, azone derivatives, propylene glycol, glycolic derivatives, cyclodextrins, octylsalicylate, cyclopentadecanolide, polysorbates, polyvinylpyrrolidone, and polyvinylpyrrolidone derivatives.

33. The device of claim 31, wherein the at least one penetration accelerator compound is present in the matrix in an amount ranging from 0.01% to 10% by weight relative to the total weight of the matrix.

34. The device of claim 1, further comprising at least one compound chosen from gelling agents, adhesive polymers, stabilizers, preserving agents, dyes, pigments, fillers, aqueous or organic solvent media, fragrances, ions, polymerization initiators, and mixtures thereof.

35. A method for the cosmetic treatment of nails comprising applying to the nails a transungual device comprising at least one active agent chosen from extracts of a non-fruiting, non-photosynthetic filamentous bacterium, dialkyl sulphones, and mixtures thereof.

36. A method for strengthening the nails comprising applying to the nails a transungual device comprising at least one active agent chosen from extracts of a non-fruiting, non-photosynthetic filamentous bacterium, dialkyl sulphones, and mixtures thereof.

37. The method of claim 36, wherein strengthening the nails comprises hardening the nails, increasing their resistance to breaking and/or to splitting, increasing the thickness of the nails, and/or stimulating the growth and/or regrowth of the nails.

38. A method for limiting the trans epidermal water loss of the nails, improving the moisturizing of the nails, and/or improving the penetration of an active agent in the nails comprising applying to the nails a transungual device comprising at least one active agent chosen from extracts of a non-fruiting, non-photosynthetic filamentous bacterium, dialkyl sulphones, and mixtures thereof.
